(19) United States
(12) Patent Application Publication
(10) Pub. No.: US 2020/0082927 A1
(43) Pub. Date: Mar. 12, 2020

(54) PLATFORM FOR DELIVERING DIGITAL BEHAVIOR THERAPIES TO PATIENTS

(71) Applicant: Enlyte Inc., Seattle, WA (US)

(72) Inventor: Donald Charles Hernandez, Seattle, WA (US)

(21) Appl. No.: 16/432,792

(22) Filed: Jun. 5, 2019

Related U.S. Application Data
(60) Provisional application No. 62/746,176, filed on Oct. 16, 2018.
(60) Provisional application No. 62/730,522, filed on Sep. 12, 2018.

(51) Int. Cl. G16H 20/70; A61B 5/0478; A61B 5/16; A61B 5/048; A61M 21/02; G16H 80/00

(52) U.S. Cl. CPC G16H 20/70; A61B 5/0478; A61B 5/165; A61M 2021/0027; A61B 5/168; A61M 21/02; G16H 80/00; A61B 5/048

(57) ABSTRACT

A technological platform for delivering supervised and unsupervised digital behavior therapies to patients suffering from any one of a variety of maladies, including but not limited to stress, anxiety, addiction and depression. The platform includes a server computer (110), a database (120), a mobile patient device (150), and a therapist device (160). The mobile patient device comprises a processor, memory, an audio system, and a mobile app (the “WithHope” app) that configures the mobile patient device to deliver selected behavior therapies to a patient, including a selected binaural beat designed to induce a selected brain wave in the patient in combination with a selected set of subliminal affirmations. Alternatively, the mobile patient device could be configured to deliver a selected binaural beat designed to induce a selected brain wave in the patient in combination with exercises in meditation.

### CROSS REFERENCE

This application claims priority to U.S. Provisional Application No. 62/846,176, “PLATFORM FOR DELIVERING DIGITAL BEHAVIOR THERAPIES TO PATIENTS,” filed on May 10, 2019, and U.S. Provisional Application No. 62/730,522, “Binaural Beats and Subliminal Suggestions,” filed on Sep. 12, 2018, which are hereby incorporated by reference in their entirety.

### TECHNICAL FIELD

The present disclosure relates generally to the field of digital therapeutics, and more particularly to a platform for providing supervised and unsupervised digital behavior therapies to patients suffering from mind-body maladies.

### BACKGROUND

The mental health crisis costs companies around the world over $1 Trillion in lost productivity and increased health care insurance premiums annually. The productivity losses are primarily caused by absenteeism, and turnover and replacement costs. In addition, the costs attributable to the family members and loved ones of employees (employee ecosystem) cost employers approximately 250% more in lost productivity than their direct employees. According to a report from the World Health Organization (WHO), 450 million people are currently suffering from mental health disorders leading to illness and disability¹. The Lancet Commission on mental health predicts that by 2030, mental illness will cost the world USD 16 trillion². If we look at the US alone, 40.3 million people are affected by the disease of addiction. Twenty percent of US deaths are attributed to addiction to tobacco, alcohol, drugs and other substances.

While many companies have Employee Assistance Programs in place that cover mental health issues for their employees and close family members, there is a strong reluctance amongst those covered to use the available benefits because of the stigma attached to mental health issues and the fear that the employee's sensitive personal information may be accessed by the employer. Stigma is the key reason preventing many people that urgently need help from receiving it. In addition, treatment facility capacity is another reason that people are not getting the help they need. There needs to be increased access to evidence-based treatment options for the public³.

### SUMMARY

The present invention relates to a platform for providing supervised and unsupervised digital behavior therapies to patients suffering from mind-body maladies including but not limited to stress, anxiety, addiction and depression, self-harm, suicidal ideation and PTSD. The inventive platform integrates behavioral science approaches, including inducing selected brain waves in patients using binaural beats and providing subliminal auditory communications, into a digital platform that enables delivery of behavior-based therapies more cost-effectively and over a wider range of patients.

In an illustrative embodiment, the inventive platform include, a server computer 110, a database 120, a mobile patient device 150, and a therapist device 160 (see FIG. 1). The mobile patient device comprises a processor, memory, an audio system, and a mobile app (the “WithHope” app) that configures the mobile patient device to deliver selected behavior therapies to a patient, including a selected binaural beat designed to induce a selected brain wave in the patient in combination with a selected set of subliminal affirmations.

The binaural beats themselves are not sufficient to help a user or patient with an affliction, since the beats merely induce a brain state conducive to what the user/patient wants at the time. It is the subliminal affirmations layered with the binaural beats that are used to help people better cope with the issues they are struggling with at a given time. Example of the subliminal affirmations are provided below, and all of these are preferably available with each of the brains state (binaural beat files) in the platform's media library.

Alternatively, or in addition, the mobile patient device could be configured to deliver a selected binaural beat designed to induce a selected brain wave in the patient in combination with exercises in meditation. Other features of the inventive platform are described below.

### BRIEF DESCRIPTION OF THE DRAWINGS

FIG. 1 schematically depicts an illustrative embodiment of a technological platform for delivering digital therapies to patients in accordance with the present invention.

FIG. 1A is a block diagram depicting the hardware components of the mobile patient device, and the software and data components of the inventive mobile patient app, which we call the “WithHope app.

FIGS. 2A and 2B are graphs of data showing the effectiveness of cognitive behavioral therapy (CBT) and computer based training for CBT, i.e., CBT4CBT.

FIGS. 3A-3C depict a first set of graphical user interface (GUI) screens of the patient app.

FIGS. 4A-4E depict a second set of GUI screens of the patient app.

FIGS. 5A-5D depict a third set of GUI screens of the patient app.

FIGS. 6A-4D depict a fourth set of GUI screens of the patient app.

FIGS. 7A-7E depict a fifth set of GUI screens of the patient app.

FIGS. 8A-8E depict a sixth set of GUI screens of the patient app.

FIGS. 9A-9D depict a seventh set of GUI screens of the patient app.

FIGS. 10A-10C depict an eighth set of GUI screens of the patient app.

FIGS. 11A-11H depict a ninth set of GUI screens of the patient app.

FIGS. 12A-12C depict a tenth set of GUI screens of the patient app.

### DETAILED DESCRIPTION OF ILLUSTRATIVE EMBODIMENTS

The present disclosure describes an inventive platform that can help save companies millions or hundreds of millions of dollars. Our approach follows a concept developed by Professor Dr. Falk Kiefer and Dr. Jan Malte Bumb, both governing doctors in the Central Institute of Mental Health (ZI) in Mannheim, Germany. It was built upon a solid base of cognitive behavioral therapy (CBT) to help reduce relapse rates and improve overall mental health. We have also integrated a gratitude journal, binaural beats, meditation and sleep tracking into the platform to take a more holistic approach and address wellness on every level. Because each person handles treatment and recovery in his or her own way, we are interested in providing a holistic approach as well as additional resources designed to help improve the user's mind-body health. We believe this will lead to higher long-term success and encourage participants to follow-up and complete their respective treatment plans.

The combination of binaural beats and subliminal affirmations can be used to help both individuals and companies by increasing wellness and decreasing productivity losses due to mental health issues. On an individual level, this will greatly increase the effectiveness of treatment for a variety of mental health conditions, including but not limited to: stress, eating disorders, anxiety, PTSD, and depression. The inventive platform can also be used to prevent relapse into any form of addiction (nicotine, alcohol, gambling, sex, internet and media, illicit drugs of all types, and legal, controlled medications of all types), as well as aid in the elimination of suicide ideation and support suicide prevention. The platform will also benefit employers by helping to increase workforce productivity through reduced absenteeism, turnover and replacement costs (both voluntary and involuntary) as well as decrease healthcare premiums. In addition, the platform provides employers with the ability to record personalized affirmations to reinforce certain key performance indicators for specific groups to improve corporate performance.

Platform Technology

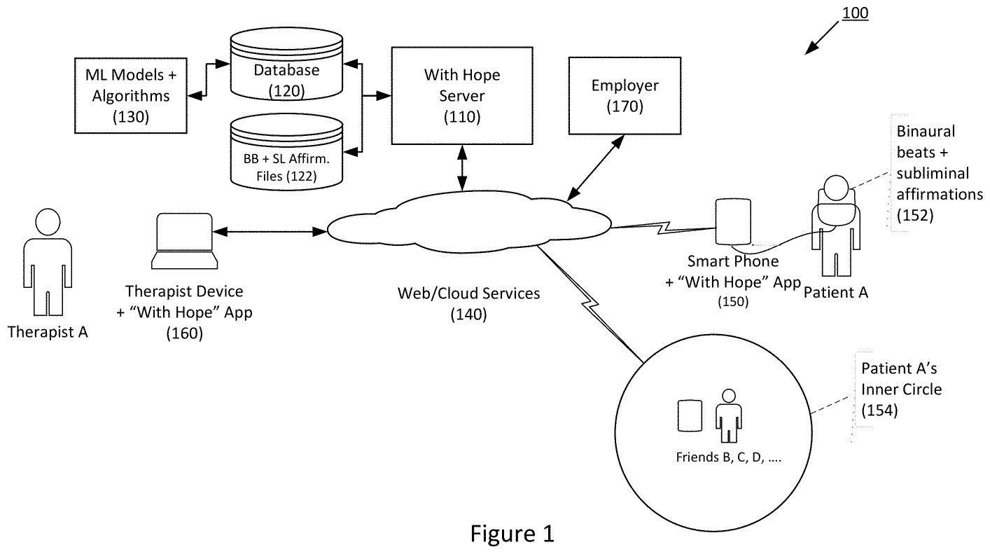

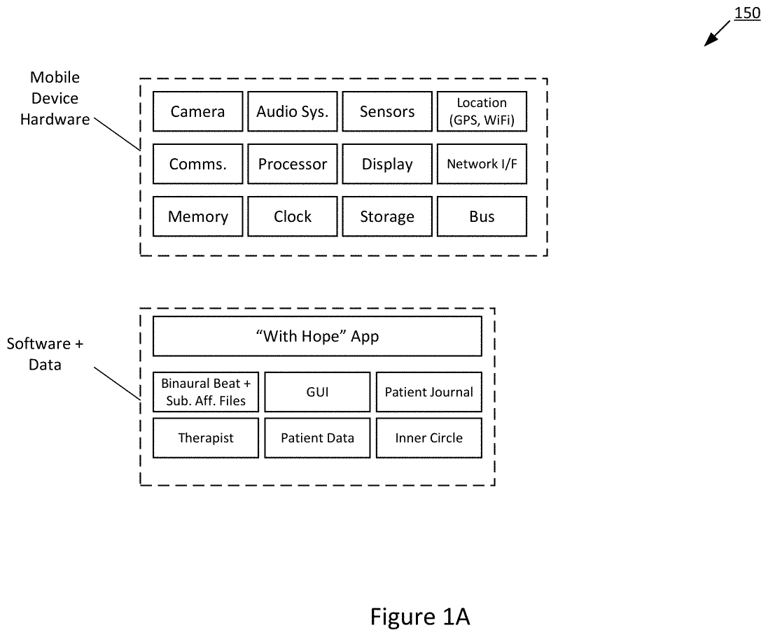

FIG. 1 depicts an illustrative embodiment of a platform 100 for delivering digital therapies in accordance with the present invention. As shown, the platform 100 includes a server computer 110, which we call the “WithHope” server. The server 110 is coupled to a database 120 and a storage area 122 for storing audio files relating to the binaural beats and subliminal affirmation messages employed for certain therapies. These files may be compressed for storage and transmission purposes but are designed for playback by the patient's mobile device. Accordingly, the audio files may be downloaded as uncompressed audio files (e.g., where the patient device is not equipped to decompress the files) or downloaded in compressed form and decompressed and stored locally by the patient device.

As discussed below, the platform may be equipped with machine learning models and algorithms, denoted by reference numeral 130, trained to learn from the patient data stored in the database 120 in order to improve upon the therapies offered to each patient.

The “WithHope” server 110 is coupled to a network 140 as a web service. In this way, the WithHope server can be communicatively coupled with the patient's smart phone including the “WithHope” patient app 150. One or more therapist devices 160 (also including the “WithHope” app, a portal for employers 170, and an inner circle 154 of friends or family members for each patient may also be included on the platform 100. The treatment facility or therapist can use the dashboard to easily see relapse or self-destructive deviations in behavior of the patient. Without this feature, the therapist would had no visibility into the wellness of a patient once the patient leaves treatment. This provides the opportunity for therapists to provide pro-active after care for patients, thus increasing their revenue generation opportunities while at the same time preventing destructive behavior in patients no longer in their care.

FIG. 1A is a block diagram depicting the hardware components of the mobile patient device, and the software and data components of the inventive mobile patient app, which we call the “WithHope” app. FIG. 1A illustrates several components of an exemplary patient device 150 in accordance with one embodiment. In various embodiments, patient device 150 will preferably include a mobile device, such as a smartphone, but may also include a desktop PC, workstation, laptop, tablet, appliance, or other computing device that is capable of performing operations such as those described herein. In some embodiments, patient device 150 may include many more components than those shown in FIG. 1A. However, it is not necessary that all of these generally conventional components be shown in order to disclose an illustrative embodiment. Collectively, the various tangible components or a subset of the tangible components may be referred to herein as “logic” configured or adapted in a particular way, for example as logic configured or adapted with particular software or firmware. In FIG. 1A, we show the mobile device hardware components separately from the components of the “WithHope” app.

As shown, the hardware components include a processor, memory, clock, and storage, as well as a camera, audio system (including an audio output for headphones/earbuds), sensors, and a location subsystem (e.g., a GPS or Wi-Fi based location system). The components of the “WithHope” app include the software and data to enable the binaural beats plus subliminal affirmations as discussed herein. In addition, the graphical user interface (GUI), patient journal, therapist data, patient data, and inner circle data are shown.

The AI and Machine Learning component of WithHope will aggregate data from multiple sources, including but not limited to mental health treatment facility databases, national and global organizations, and the patients using the WithHope platform. Deviations in a patient's behavior will also be collected. This information will be used as a body of evidence to provide a proactive interaction in case of negative deviation in a patient's behavior. The particular algorithms used will be updated regularly to ensure optimal accuracy and efficiency.

In various embodiments, patient device 150 may comprise one or more physical and/or logical devices that collectively provide the functionalities described herein. In some embodiments, patient device 150 may comprise one or more replicated and/or distributed physical or logical devices. In some embodiments, the patient device 150 may comprise one or more computing resources provisioned from a “cloud computing” provider. In addition, a typical patient device 150 will include a bus interconnecting several components including a network interface, display, central processing unit, and a memory. The memory generally comprises a random access memory (“RAM”), and the storage generally comprises a permanent non-transitory mass storage device, such as a hard disk drive or solid-state drive. An operating system (not shown) will also be present in memory.

Binaural Beats+Subliminal Affirmations

Binaural beats are created by presenting one tone to each ear separately with slightly different frequencies, resulting in a “binaural beat” that induces a certain brain state. The binaural beat is created by the brain, and the effectiveness of creating binaural beats has been proven with a wide body of scientific research originating in Prussia in 1839, and continuing today. An article in Scientific American by Gerald Oster in 1973 is credited with bringing this to modern attention and renewed research. This is supplemented with subliminal affirmations to help with specific behavioral health challenges, but there is a lack of scientific evidence to prove the effectiveness of subliminal affirmations. However, what has been proven is that the placebo effect with subliminal affirmations is quite powerful in its own right.

Binaural beats are produced within the brain, and the rhythm of the binaural beats equals the difference between the two tones. For example, when 410 Hz is presented to the right ear and 400 Hz is presented to the left year, a binaural beat of 10 Hz is perceived. If sustained, the binaural beats can be measured in the cerebral cortex via electroencephalographic (EEG) measurements. The frequency of binaural beats could therefore be selected to produce EEG-associated states.

Cerebral activity recorded by EEG is divided into the following categories: Delta waves (0.5-3 Hz, deepest meditation and dreamless sleep), Theta waves (3-8 Hz, Occurs in sleep, deep meditation), Alpha waves (8-12 Hz, power of now, being in the present, aids in mental coordination, calmness, mind/body integration), Beta waves (12-38 Hz, Waking state of consciousness when attention is directed to the outside world and cognitive thoughts), and Gamma waves (38-42 Hz, Cognitive functioning, tasks that require higher processing, learning information, memory)¹⁶.

There is growing research and support suggesting that binaural beats indeed affect cognition and psychophysiological states. Binaural beats have been used to induce brain wave states in the past to decrease anxiety¹⁷. A study of approximately 100 pre-operative anxiety patients measured the use of binaural beats in treating the anxiety level of the participants and produced a 26.3% reduction in the anxiety level of the participants¹⁸. A similar study of pre-operative patients listening to binaural beats with differing music found decreased levels of anxiety without regard to the music being played. However, their anxiety levels were lowest immediately following the listening period¹⁹.

The use of binaural beats in the treatment of other mental illnesses and conditions. In a study by Ellen Saxby and Eugene Peniston used binaural beats; a method of brain entrainment, to validate their effectiveness in reducing depression and recidivism rates in alcoholics²⁰. Their study found the participants had a sharp reduction in their self-diagnosed depression and in a follow-up twenty-one months later found a sustained prevention of relapse. There was a follow-up study that showed the same levels of trait anxiety 18 months post the study giving additional support to the long-range effectiveness of the treatment²¹.

The US has a significant issue in dealing with people suffering with Post Traumatic Stress Disorder (PTSD). The annual cost to society of anxiety disorders is estimated to be $42.3 billion (in 1990 dollars), often due to misdiagnosis and undertreatment²². There has been limited research to date, but some positive outcomes have been detected. Work performed by George Lindfield in working with more than 60 PTSD patients over a 5-year period showed success²⁴. Although additional research needs to be completed to meet the requirements of the Veterans Administration, the outcome has been promising.

Meditation can help reduce stress and acts as a gentle reminder to be mindful. Taking some time to relax and listening to calming music that is infused with binaural beats (theta waves) will ease you into meditation.

In addition, patients will also have the option of listening to binaural beats with subliminal messages²³. We will present two tones to each ear separately with slightly different frequencies. It results in an auditory illusion, which occurs inside the brain. These are binaural beats. Positive affirmations are added subliminally to the track to overcome negative thoughts and be more optimistic.

As discussed above, it's not the binaural beats themselves that help with affliction, as they induce a brain state that is conducive to what the user/patient wants at the time. For example, a given user may listen to Theta to meditate upon waking up in the morning, Gamma during the day when working, and Delta when going to sleep. It is the subliminal affirmations that are layered with the binaural beats that are used to help people better cope with the issues they are struggling with. Examples of these are listed below, and all of these (stress, anxiety, depression, addiction (SUD), eating, smoking, and self-harm coupled with suicidal ideation), are available with each of the brains states in the platform's media library.

The selection process steps include:

1) Select binaural beats.

2) Select brain state.

3) Select one of the seven issues that a person would like help with.

In other words, users/patients can mix and match the brain state and subliminal affirmations to provide them exactly what they need at the time.

In addition, the titles of the subliminal affirmations can be changed regularly as needed, e.g., to make them sound more suitable. For example, the following titles may be used: “Help with My Eating”; “End My Anxiety”; “Help with My Addictions”; “Make Me Happy”, etc.

An example set of working titles for the binaural beats includes:

Peak Performance (Gamma)

Develop inspiration, rapid memory recall, and a peak performance mindset.

Alert and Attentive (Beta)

Develop a focused problem solving and decision making attitude.

Boundless Creativity (Alpha)

Improve imagination, creative problem solving and intuition.

Sweet Meditation (Theta)

Improve your meditation practice, increase focus, calmness and happiness.

Deep, Dreamless Sleep (Delta)

Ease into a Deep Sleep and improve your ability to learn quicker.

An example set of positive affirmations are shown below for a number of afflictions, including anxiety, stress, depression, substance use disorder (SUD), eating disorders, smoking cessation, and suicidal ideation.

Anxiety

1. I am calm and collected

2. I am confident that I will overcome life's problems

3. I am social and friendly

4. I look forward to meeting new people

5. With every breath, I release my anxiety and tension

6. All is well and I am safe

7. I overcome every situation and my fears with courage

8. I have confidence in myself and my abilities to overcome challenges

9. My body is relaxed

10. My mind is relaxed and calm

11. My mind is clear and filled with positive thoughts

12. I believe in myself

13. I know change is constant and I am prepared for it

14. I always accomplish my goals

15. I live in peace

16. The future looks good

17. I look forward to my future WithHope and happiness

18. I am free of anxiety

19. I am free of stress

20. Life is wonderful

21. I love myself deeply and unconditionally

Stress

1. I am relaxed

2. I am calm and collected

3. With every breadth, I release my stress and tension

4. My body is relaxed

5. My worries are melting away

6. I believe in myself

7. I have confidence in myself and my abilities to overcome challenges

8. I always accomplish my goals

9. My future looks good

10. I look forward to the future WithHope and happiness

11. I overcome every situation and my fears with courage

12. I am stress free

13. I appreciate myself

14. I am proud of who I am

15. I love myself deeply and unconditionally

16. My mind is relaxed and calm

17. My mind is clear and filled with positive thoughts

18. I am happy

19. I live in peace

20. Life is wonderful

Depression

1. I am happy

2. I am calm

3. I am positive and live a fulfilling life

4. With each day, I am more positive and happy

5. With every breath, I relax my mind

6. My mind is releasing all the tension and it is clear

7. My mind is clear and is filled with positive thoughts

8. I solve life's problems with courage

9. I overcome my fears and doubts with confidence

10. I have confidence in myself

11. I am social and friendly

12. Change is constant and I am prepared for it

13. My worries are melting away

14. My body is relaxed

15. Life is wonderful

16. I love myself deeply and unconditionally

17. I look forward to my future WithHope and happiness

18. I am proud of who I am

19. I appreciate myself

20. I am living in peace

SUD

1. I am relaxed and I am free of stress, fear and anxiety

2. I am free and I can manage my stress, fear and anxiety

3. I can resist my temptation

4. I am stronger than any substance

5. I am highly motivated and I am in control of my urges and impulses

6. I am in control of my thoughts and actions

7. I surround myself with people who are supportive

8. My body is relaxed

9. My worries are melting away

10. I have been doing great and I improve everyday

11. I am proud of who I am

12. I am living in peace

13. I am confident and I overcome my doubts

14. I believe in myself

15. I appreciate myself

16. I live a fulfilling life

17. I deserve to be happy and healthy

18. I love myself deeply and unconditionally

19. I embrace changes to lead a healthy lifestyle

20. I am calm and composed

Eating

1. I love my body

2. I enjoy being fit and healthy

3. I am getting better everyday

4. I eat a healthy diet

5. I eat a balanced diet

6. I embrace changes to lead a healthy lifestyle

7. I love myself deeply and unconditionally

8. I believe in myself

9. I appreciate myself

10. I am proud of myself

11. I am active and exercise regularly

12. Exercise is a part of my daily routine

13. I surround myself with people who are supportive

14. My motivation to be healthy is very high

15. My worries are melting away

16. My thoughts are slowing down

17. I am relaxed and I am free of stress, fear and anxiety

18. I am free and I can manage my stress, fear and anxiety

19. I am happy

20. My mind is filled with positive thoughts

Smoking Cessation

1. I am relaxed and I am free of stress, fear and anxiety

2. I am free and I can manage my stress, fear and anxiety

3. I can easily resist any temptation

4. My worries are melting away

5. I can easily resist my urge to smoke

6. I enjoy being fit and healthy

7. My lungs are healthy

8. I surround myself with people who are supportive

9. I can live without cigarettes

10. I save money by not smoking

11. My food tastes better

12. I am confident I can overcome this habit

13. I am confident I can overcome life's problems

14. I am proud of who I am

15. I am living in peace

16. My body is relaxed

17. My mind is relaxed and calm

18. I am positive and my mind is filled with happy thoughts

19. I love myself deeply and unconditionally

20. I am happy

Suicidal Ideation

1. I am cool, calm and collected

2. I am at living in peace

3. I am happy

4. I love myself deeply and unconditionally

5. I look forward to my future WithHope and happiness

6. I am positive and my mind is filled with happy thoughts

7. Life is wonderful

8. The world is beautiful

9. I have a great future

10. I believe in myself

11. I appreciate myself

12. I am proud of who I am

13. My body is relaxed

14. My mind is relaxed, clear and calm

15. My life is worth a lot

16. With each day, my happiness and positive thoughts increase

17. I feel good about myself and everything around me

18. I feel healthy and fit

19. I am a good person

20. I am a friendly person

Cognitive Behavioral Therapy

Cognitive behavioral therapy (CBT) is a type of psychotherapy. The father of CBT, Aaron T. Beck, realized that there is a strong link between our thoughts and feelings. The goal of Cognitive Based Therapy (CBT) is to change how a person evaluates his/her thoughts and assumptions. So the way a person feels is linked to how they think about a situation or how they perceive a situation⁴. This self-awareness could help change behavior, reduce substance use, and adopt a healthy lifestyle, and thereby improving quality of life, happiness and productivity.

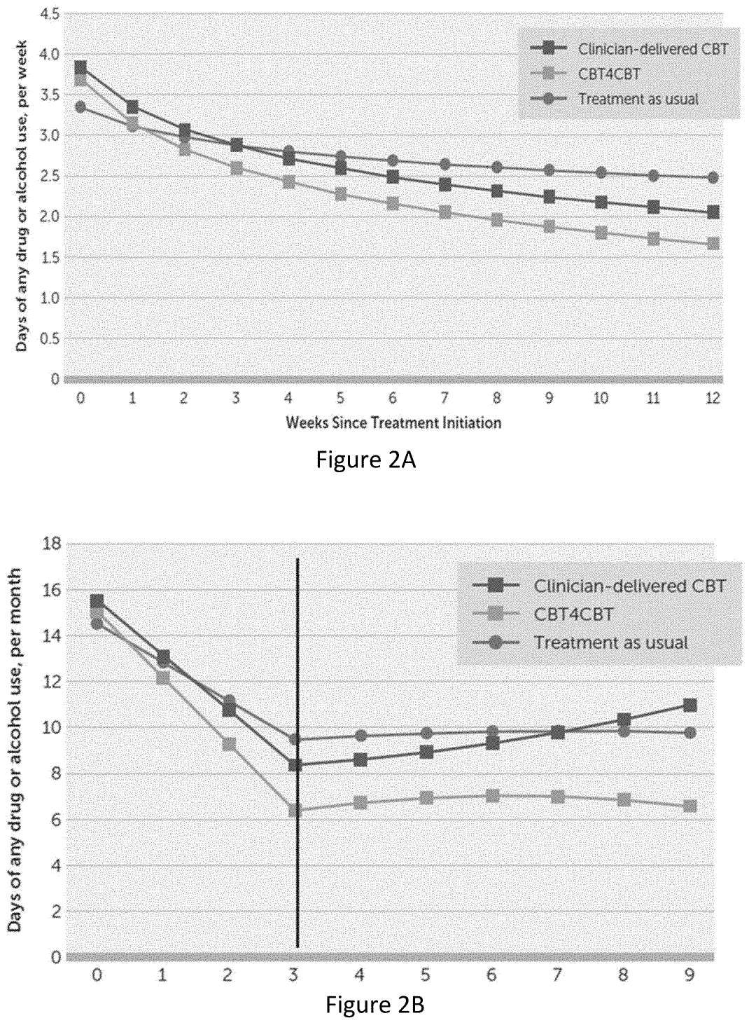

There is limited availability, uptake and fidelity of evidence-based treatments. Therefore, there is growing interest in web-based interventions, which can provide greater accessibility and standardization as well as potential cost savings. Meta-analysis suggests that there is a significant but modest effect of these approaches in decreasing stress, anxiety, depression, and addiction. To overcome the limitations in some of the studies, Kiluk et al 2018, compared clinician based, CBT and Computer Based Training CBT (CBT4CBT) with standard outpatient treatment (treatment as usual) for substance use disorders⁵. See FIGS. 2A and 2B.
Treatment retention was significantly higher in the CBT4CBT condition, lowest in the clinician-delivered CBT condition and intermediate in the treatment as usual condition.
Participants, who received CBT4CBT or CBT, reduced their frequency of substance use significantly more than those who received treatment as usual.
Six-month follow-up outcomes indicated continuing benefit of CBT4CBT over treatment as usual, but not for clinician-delivered CBT over treatment as usual.
Participants in the CBT4CBT group demonstrated the best learning of cognitive and behavioral concepts, as well as the highest satisfaction with treatment.
In sum, this study provides strong support for CBT4CBT as an efficacious treatment for substance use, even when offered with limited clinical contact. Web-based CBT4CBT will not only broaden access to an evidence-based treatment, but it also may be a more appealing option for many individuals.

Charting and Journaling

By charting moods, activities and writing down thoughts (journals), one learns how to identify and evaluate experiences and their consequences—for example, whether the experience being captured makes one upset, crave drugs or alcohol, or think of suicide. In the illustrative embodiment, the platform presents the data in such a way that users of the platform are more able to spot trends and avert negative outcomes.

The way people interpret situations might not always be accurate. You might feel something is true, but in reality, it might not be true. Examples:

1) A small setback feels like the worst thing ever.

2) You try to predict the future and how it will be bad.

3) You try to read someone else's mind without having enough information.

Another Example: For a person who has undergone trauma, a situation, person or a news story could remind them about the trauma leading to fear. The trauma could change one's experience and belief in oneself, others and society. Changing these associations, assumptions and beliefs would be the goal of treating someone with PTSD.

Once you learn to identify thoughts and moods, one can begin to apply the cognitive restructuring technique. The platform teaches people how to create new thoughts for the situation, therefore changing their feelings and behavior for the same situation. The cognitive restructuring technique helps people cope with distressing thoughts and manage the situation better.

Cravings, Triggers and Consumption

For addiction concerns, the inventive platform also allows users to record, recognize and evaluate triggers and cravings in a similar fashion and presents trends to help one be more mindful. The inventive platform will enable people to identify common triggers and identify correlations with moods and activities, thus helping better plan their day in a constructive way.

Gratitude Journal

There is a growing interest in positive psychological interventions in recent years and gratitude practice has received a lot of attention. The possibility of including gratitude intervention in psychotherapy is being assessed. What is gratitude? Gratitude is simply appreciating what is valuable and meaningful in one's life, providing a feeling of thankfulness and appreciation.

Robert Emmons, a leading researcher in gratitude believes that gratitude has two key components. First, it helps affirm the good things in one's life and second, it helps identify the source of the goodness6. Sociologist George Simmel believes that it helps strengthen the bond between humans. Research suggests that gratitude could have played a significant role in human evolution and gratitude is ubiquitous and influenced by culture and society⁷. Theoretically, we can all agree that gratitude leads to a positive mindset thereby enhancing personal wellbeing. Numerous studies confirm this association as well^{6,7,8}. Patients counting their blessings and logging them in gratitude journals or writing gratitude letters can help increase wellbeing and life satisfaction. Higher levels of gratitude could also lead to better quality of sleep. A study conducted in Taiwan with athletes showed that gratitude helps improve self-esteem⁹. More research will help understand the full potential of gratitude practice and how it could be more effectively integrated into psychotherapy.

In a research study conducted, University students seeking psychotherapy sessions were divided into two groups. One group was asked to write down gratitude letters along with their psychotherapy sessions. The control group either received just psychotherapy sessions or were additionally asked to write down their experiences. It was found that students who were asked to write down gratitude letters along with their psychotherapy sessions, over time, had significantly better mental health. The results showed that using positive words leads to better wellbeing. There is emerging evidence indicating that gratitude practice can help alleviate stress, depression and post-traumatic stress disorder¹⁰. Further studies have to be conducted to understand which patients would benefit from gratitude interventions.

In everyone's day-to-day life with all the accompanying trials and tribulations, the mind can forget to focus on the good things that happen in life. For example, thinking errors can lead to negative self-imagery and filtering out the good experiences. In an illustrative embodiment, the platform comes with a gratitude journal that allows one to develop a solid gratitude practice. It also comes with a list of prompts to help one savor the experiences, people and social interactions life, which are often overclouded by a predisposition to a negative bias. The analytics provided in the platform that allow correlations between the mood chart and gratitude journal will provide insight into day-to-day experiences.

Meditation

Over the years, the combination of meditation and mindfulness has become a popular wellness trend and are buzzwords that are used often. Traditionally meditation was taught in formal centers. But now, everyone has access to multiple online apps, courses and retreats, and millions of people across North America and Europe meditate regularly. It is being taught in schools and prisons, to athletes and those in the U.S Army. It is practiced by executives and employees from Silicon Valley to Wall Street. Meditation and mindfulness are also being suggested as possible adjuvant treatments for stress, depression, chronic pain, anxiety, addiction and high blood pressure.

What is mindfulness? “It has been defined as the intentional, accepting and non-judgmental focus of one's attention on the emotions, thoughts and sensations occurring in the present moment.¹¹” This state can be achieved through training and techniques such as meditation. Meditation and mindfulness can be a practical way to overcome stress and respond better to a given situation. Of course, it requires a lot of practice like any other skill, along with commitment and discipline. And it has to become a part of one's everyday routine in order to produce long term benefits.

Since the early 2000s, multiple studies have been carried out by scientists to understand what changes occur in the brain during meditation and how it could help with improving mental wellness. Studies suggest that meditation can bring about structural and functional changes in the regions of the brain that are involved in self-awareness, attention and emotion¹².

There are billions of neurons in the brain and they are constantly communicating with each other and producing electrical signals. These electrical signals result in brain waves. The brain waves are classified based on their bandwidths, which correspond to certain activities and functions, depending on the abundance and location. With the help of electrodes and wires attached to the brain, it is possible to detect these brain waves. Richard Davidson, a prominent psychologist, in a 2004 study observed gamma waves in the brains of Buddhist monks who were expert meditators, even when they were not meditating¹³. These gamma waves are associated with flash insights, peak concentration and cognitive functioning¹⁴. Studies suggest increased alpha and theta brain waves, which are associated with simple relaxation, during nondirective meditation¹⁵.

Sleep Tracking

The negative consequences of sleep deprivation are well known. According to one study, 80 million Americans are chronically sleep deprived, which means, they receive less than the recommended 7 hours of sleep at night. Our changing lifestyle, floodlit cities, use of cellphones and laptops, along with factors such as stress and depression, have made sleep deprivation an epidemic. Sleep is important for physical and mental wellness²⁴. Lack of adequate sleep can contribute to accidents and affect productivity. It can also affect our health and increase risk to dementia, heart disease and diabetes.

A sleep cycle lasts for 90 minutes and an adult completes 4-5 sleep cycles per night. As we go through the different stages of sleep cycle, our brain performs certain housekeeping functions and reinforces memories. There is growing evidence indicating sleep helps maintain blood pressure, body temperature and a healthy immune system. High levels of stress can lead to sleep disorder. At the same time, a good night's sleep can help decrease stress. Studies suggest that lack of sleep costs United States $441 billion per year. There is an increase in the use of sleeping aid and Americans spend up to $66 billion per year for medication, sleep devices and studies. This is set to rise to $85 billion per year by 2021. Small steps can go a great way in helping us improve sleep quality. Keeping the room dark at night, blocking blue light at night time, maintaining consistent sleep times, being aware of caffeine intake, nutrition and time of exercise can help. With the inventive platform described herein, patients will be able to keep track of their sleep quality and duration.

Inner Circles

The Inner Circle is a group of no more than five people that are willing to be contacted at any time to provide help and assistance when the patient is in distress. These are a group of trusted people, with whom the patient feels comfortable and can speak openly and honestly about their concerns and distress without any fears of negative bias or conviction. It provides the second to last step to keep patients from consuming in the case of substance use disorders, or self-harm and suicide.

The graphical and technical depiction of the Inner Circle consists of a circle of up to five faces, surrounding a red button connecting the patient to their country's national suicide hotline telephone number. All of the phone numbers are stored, so that all the patient has to do is touch one of the faces or red button to be connected to a life-saving contact.

User Interface

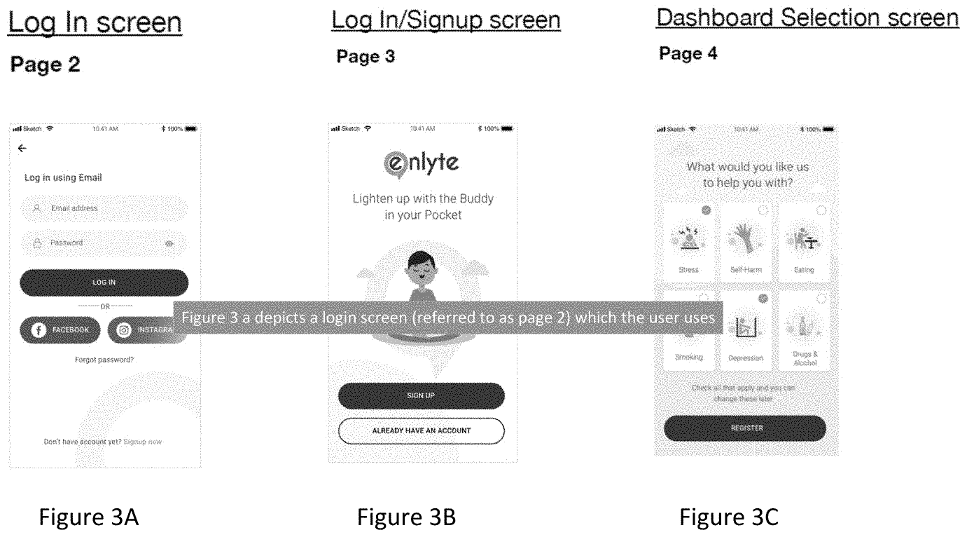

FIG. 3A depicts a login interface (page 2) of the kind a user could employ to log into the platform. This is a familiar interface of the kind in which the user can enter an email address and password, or alternatively log in using credentials from a social media platform.

FIG. 3B depicts a login/sign-up interface (page 3) of the kind a new user employ to sign up, or register, with the platform.

FIG. 3C depicts a dashboard selection interface (page 4) of the kind a user could employ to navigate to different parts of the system. For example, in this embodiment, the user can select dashboards for help with stress, self-harm, eating, smoking, depression, and drugs and alcohol.

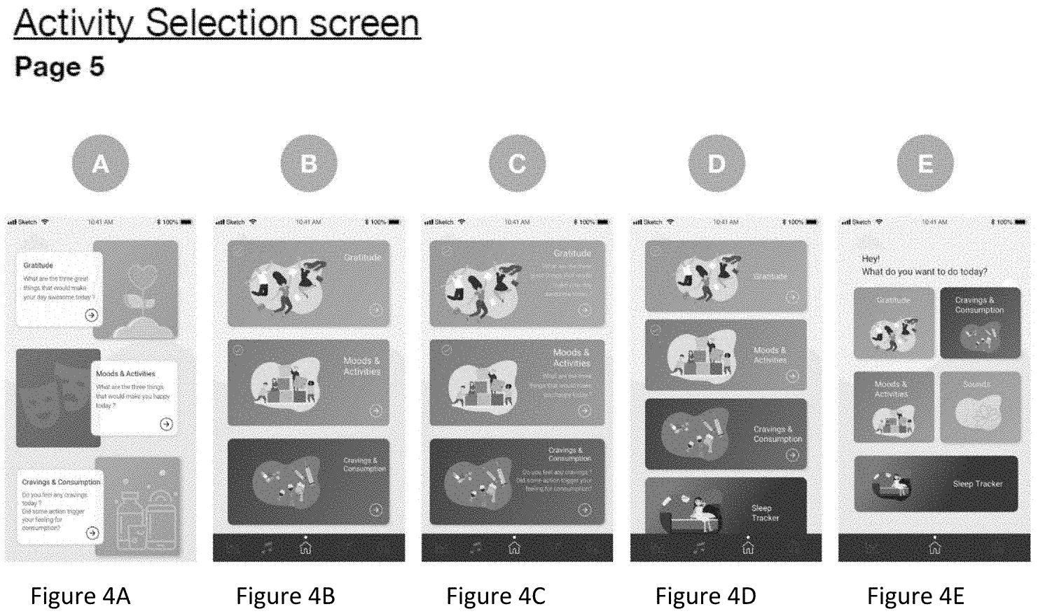

FIGS. 4A-4E (page 5) depict interfaces for navigating to resources for help with gratitude, moods and activities, and cravings and consumption.

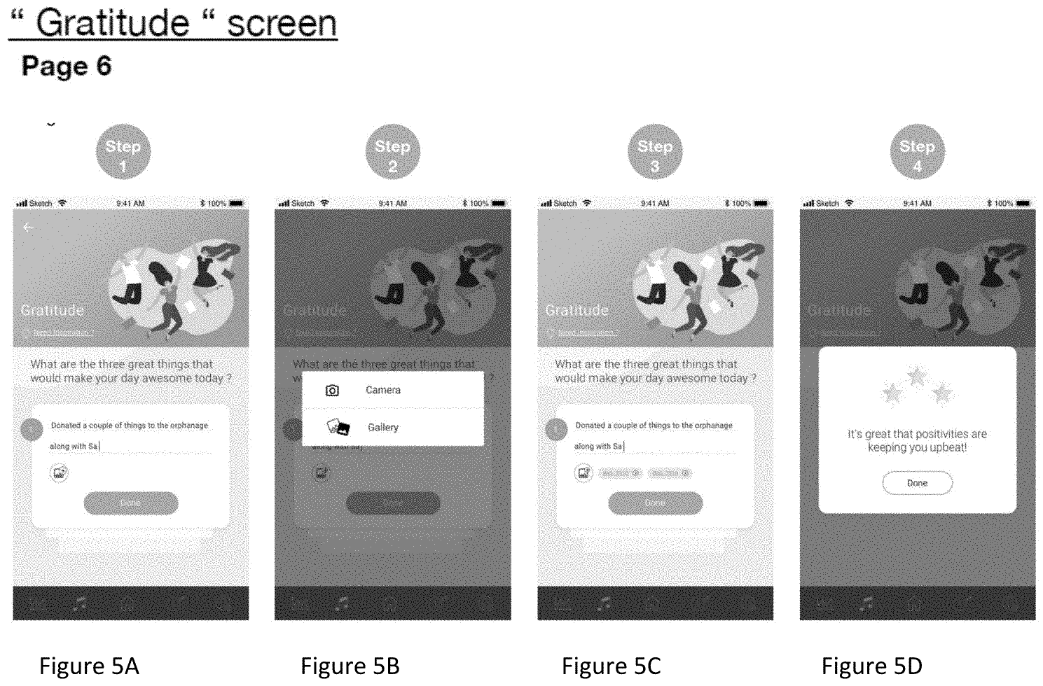

FIGS. 5A-5D (page 6) depict interfaces for inputting information into a “gratitude” journal.

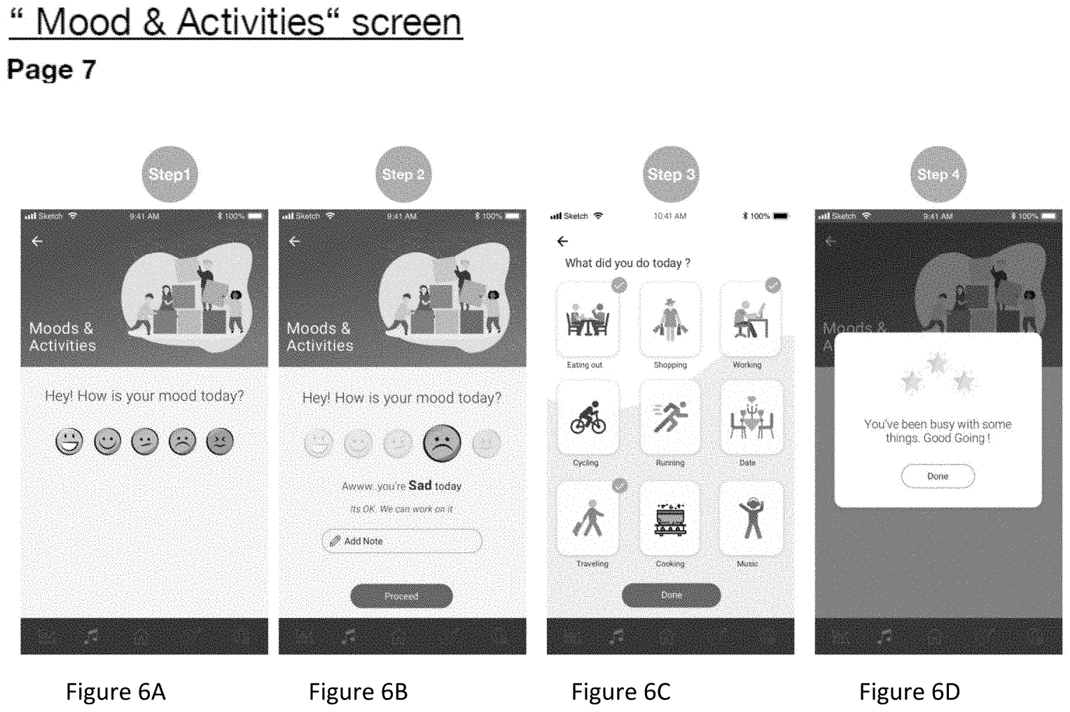

FIGS. 6A-6D (page 7) depict interfaces for inputting information into a “moods and activity” journal.

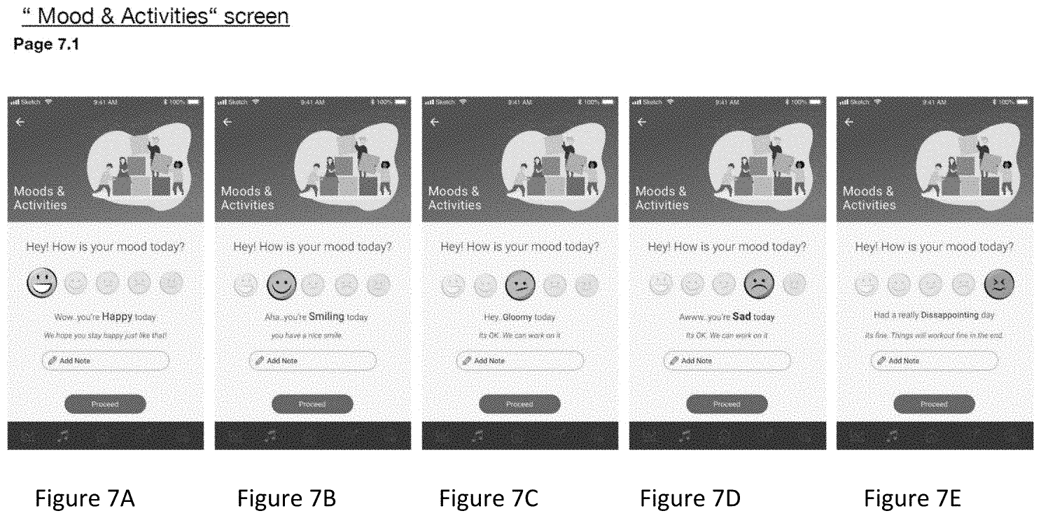

FIGS. 7A-7E (page 7.1) depict interfaces for inputting additional information into the “moods and activity” journal.

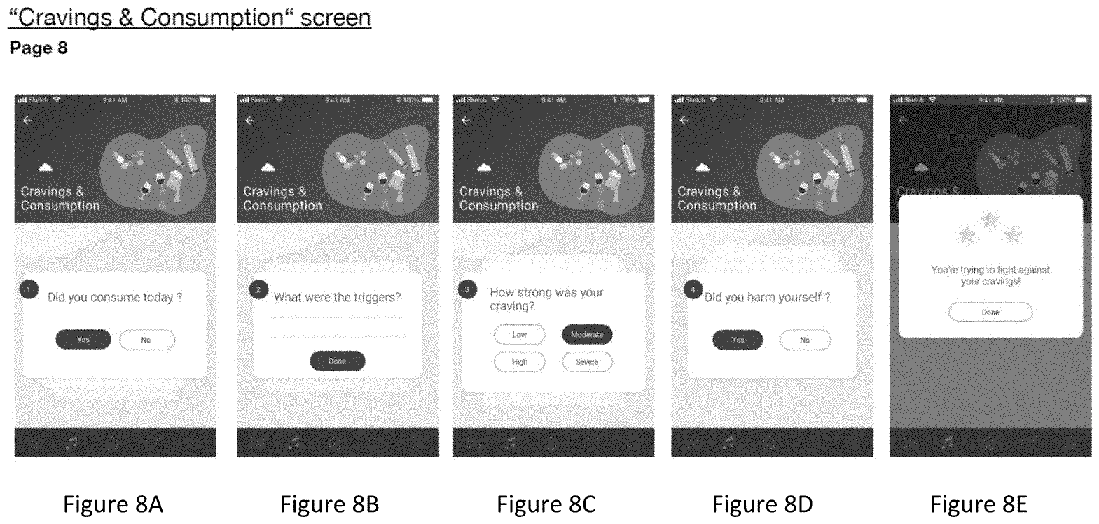

FIGS. 8A-8E (page 8) depict interfaces for inputting information into a “cravings & consumption” journal.

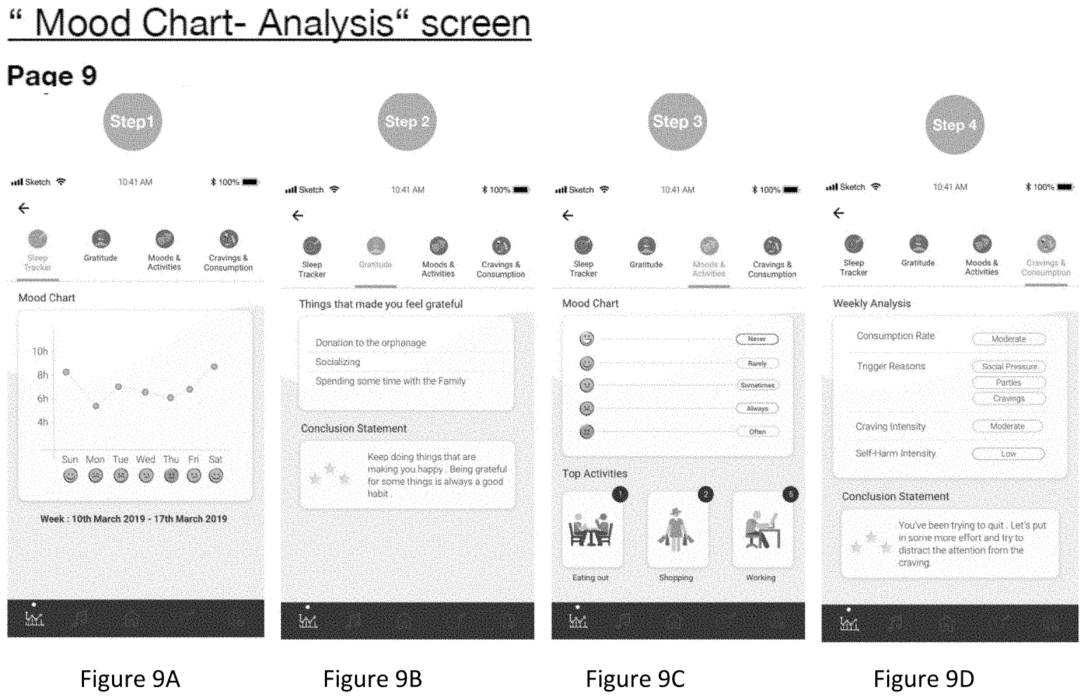

FIGS. 9A-9D (page 9) depict interfaces for displaying information on a “mood chart analysis” screen.

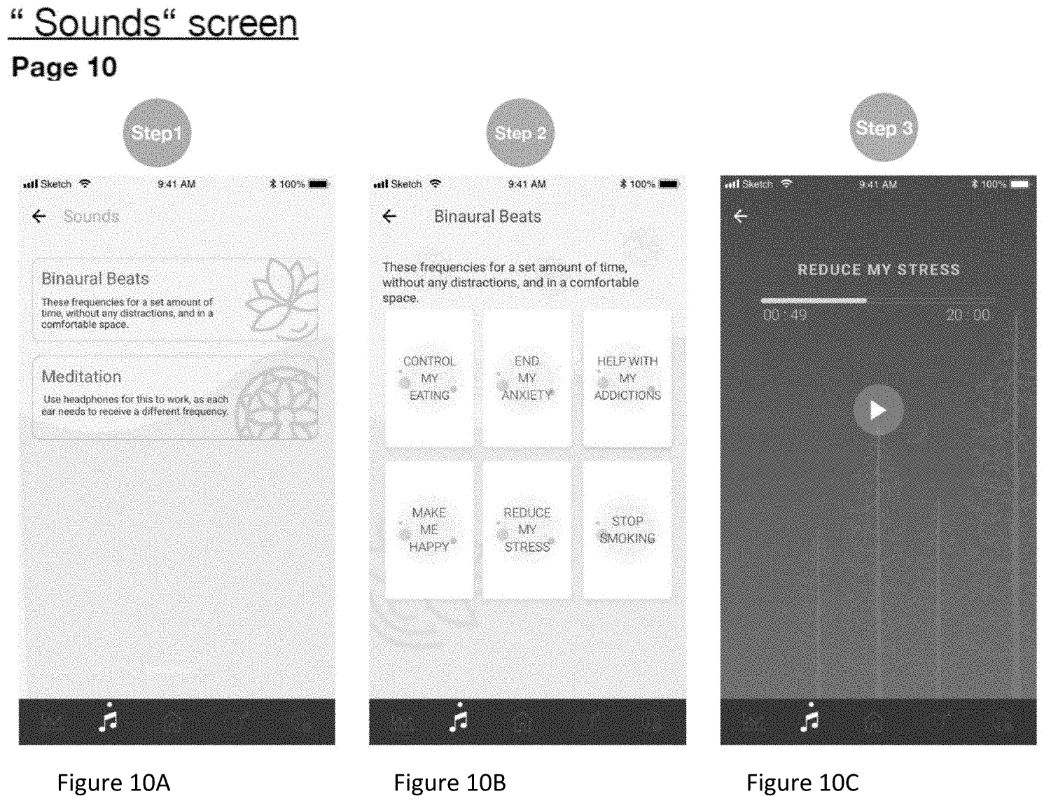

FIGS. 10A-10C (page 10) depict interfaces for accessing a “sounds” screen and binaural beats.

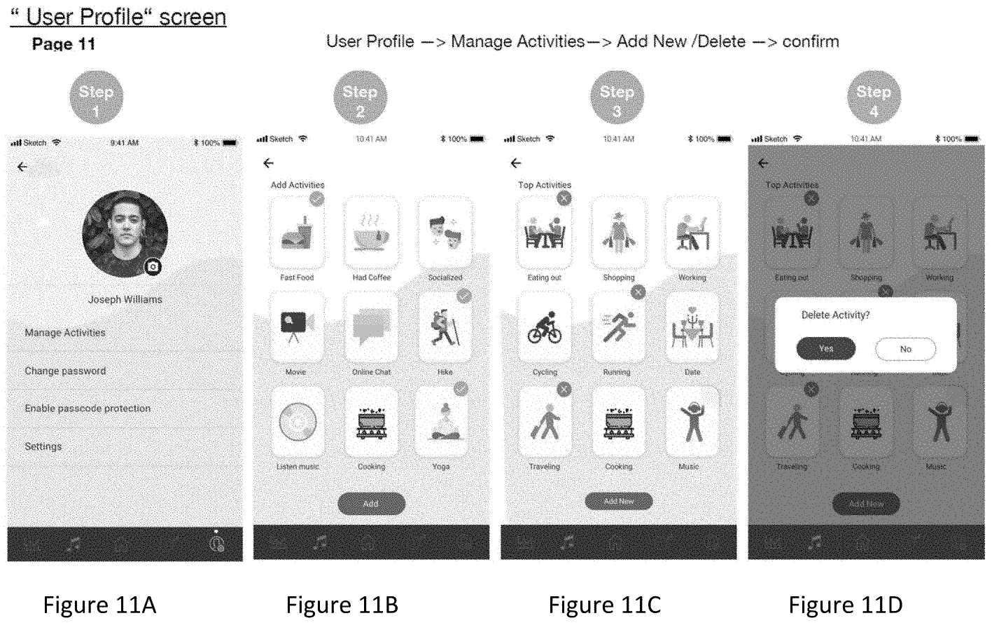

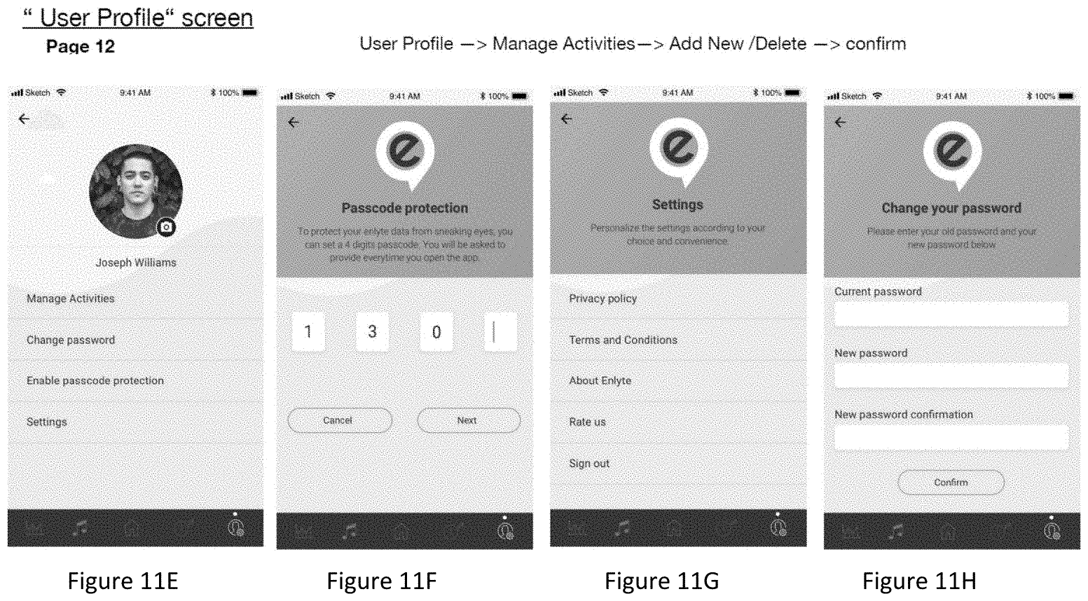

FIGS. 11A-11D (page 11) depict interfaces for inputting information into a “user profile” screen; and FIGS. 11E-11H (page 12) depict additional interfaces for the “user profile” screen.

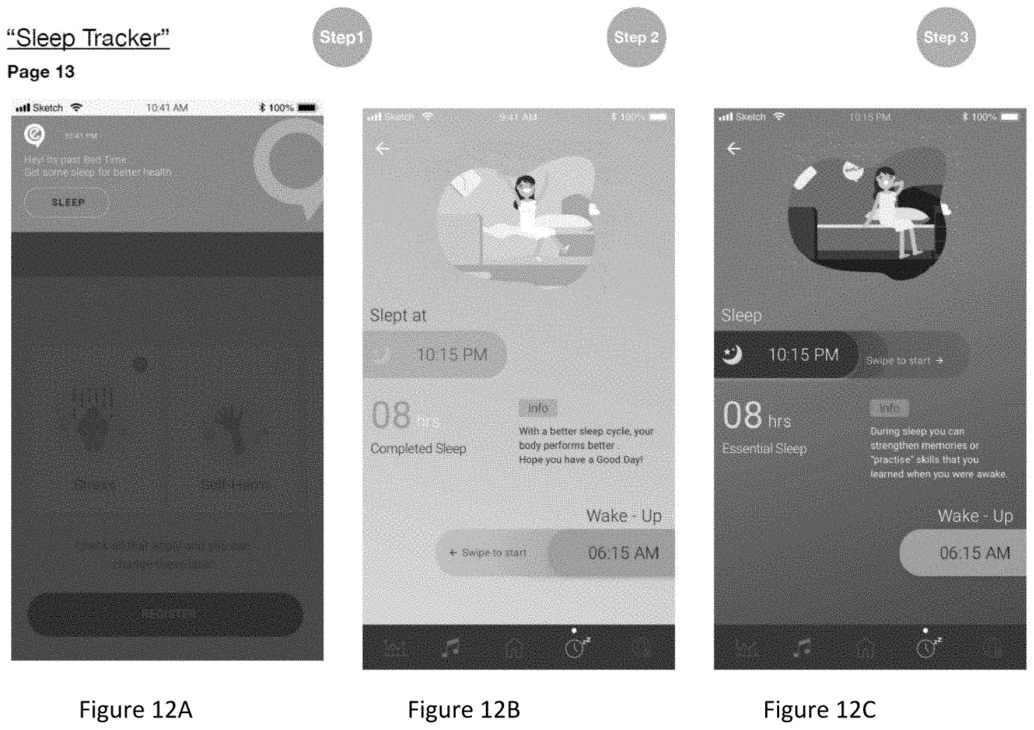

FIGS. 12A-12C (page 13) depict interfaces for a “sleep tracker”.

### CONCLUSION

We have disclosed an inventive technological platform for delivering digital behavior therapies to patients suffering from a variety of maladies. The illustrative embodiments described above are designed to provide more effective therapies to a wide range of patients that would not otherwise have access to such therapies. The inventive platform also benefits the patient's respective employers as well as their therapists.

It should be noted that the scope of protection of the following claims is by no means limited to the specific technological embodiments described above, except where the claims specifically recite such technological embodiments. For example, the inventive platform may be utilized in conjunction with various types of mobile device hardware, and with different forms of subliminal affirmations than those described above. The means and location for storage of the binaural beat and subliminal affirmation files, patient data, etc., may be changed depending upon device capabilities, bandwidth constraints, etc.

Those skilled in the art will recognize that it is common within the art to describe devices or processes in the fashion set forth herein, and thereafter use standard engineering practices to integrate such described devices or processes into larger systems. At least a portion of the devices or processes described herein can be integrated into a network processing system via a reasonable amount of experimentation. Various embodiments are described herein and presented by way of example and not limitation. Those having skill in the art will appreciate that there are various logic implementations by which processes and/or systems described herein can be effected (e.g., hardware, software, or firmware), and that the preferred vehicle will vary with the context in which the processes are deployed. If an implementer determines that speed and accuracy are paramount, the implementer may opt for a hardware or firmware implementation; alternatively, if flexibility is paramount, the implementer may opt for a solely software implementation; or, yet again alternatively, the implementer may opt for some combination of hardware, software, or firmware. Hence, there are numerous possible implementations by which the processes described herein may be effected, none of which is inherently superior to the other in that any vehicle to be utilized is a choice dependent upon the context in which the implementation will be deployed and the specific concerns (e.g., speed, flexibility, or predictability) of the implementer, any of which may vary.

Those skilled in the art will appreciate that logic may be distributed throughout one or more devices, and/or may be comprised of combinations memory, media, processing circuits and controllers, other circuits, and so on. Therefore, in the interest of clarity and correctness logic may not always be distinctly illustrated in drawings of devices and systems, although it is inherently present therein. The techniques and procedures described herein may be implemented via logic distributed in one or more computing devices. The particular distribution and choice of logic will vary according to implementation.

The foregoing detailed description has set forth various embodiments of the devices or processes via the use of block diagrams, flowcharts, or examples. Insofar as such block diagrams, flowcharts, or examples contain one or more functions or operations, it will be understood as notorious by those within the art that each function or operation within such block diagrams, flowcharts, or examples can be implemented, individually or collectively, by a wide range of hardware, software, firmware, or virtually any combination thereof. Portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in standard integrated circuits, as one or more computer programs running on one or more processing devices (e.g., as one or more programs running on one or more computer systems), as one or more programs running on one or more processors (e.g., as one or more programs running on one or more microprocessors), as firmware, or as virtually any combination thereof, and that designing the circuitry or writing the code for the software or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies equally regardless of the particular type of signal bearing media used to actually carry out the distribution. Examples of a signal bearing media include, but are not limited to, the following: recordable type media such as floppy disks, hard disk drives, CD ROMs, digital tape, flash drives, SD cards, solid state fixed or removable storage, and computer memory.

### REFERENCES

1) World Health Organization. Available from: https://www.who.int/whr/2001/media_centre/press_release/en/
2) Mental Illness Will Cost the World $16 USD Trillion by 2030. Available from: https://www.psychiatrictimes.com/mental-health/mental-illness-will-cost-world-16-usd-trillion-2030
3) Why the US Fails at Treating Addiction. Available from: https://www.livescience.com/41557-why-america-fails-at-addiction-treament.html
4) The Centre for Addiction and Mental Health. Cognitive Behavioural Therapy. Available from: https://www.camh.ca/-/media/files/guides-and-publications/cbt-guied-en.pdf
5) Kiluk B D, Nich C, Buck M B, Devore K A, Frankforter T L, LaPaglia D M et al. Randomized Clinical Trial of Computerized and Clinician-Delivered CBT in Comparison With Standard Outpatient Treatment for Substance Use Disorders: Primary Within-Treatment and Follow-Up Outcomes. Am. J Psychiatry. 2018; 175(9): 853-863
6) The Science of Gratitude. Available from: https://ggsc.berkeley.edu/images/uploads/GGSC-JTF_White_Paper-Gratitude-FINAL.pdf
7) Why gratitude is good. Available from: https://greatergood.berkeley.edu/article/item/why_gratitude_is_good
8) What can the brain reveal about gratitude? Available from: https://greatergood.berkeley.edu/article/item/what_can_the_brain_reveal_about_gratitude
9) Sansone R A, Sansone L A. Gratitude and Well Being. The Benefits of Appreciation. Psychiatry. 2010; 7(11): 18-22
10) Wong Y J, Owen J, Gabana N T, Brown J W, McInnis S, Toth P, Gilman L. Does gratitude writing improve the mental health of psychotherapy clients? Evidence from a randomized controlled trial. Psychotherapy Research. 2019; 28(2): 192-202
11) Zgierska A, Rabago D, Chawla N, Kushner K, Koehler R, Marlatt A. Mindfulness Meditation for Substance Use Disorders: A Systematic Review. Subst Abus. 2009; 30(4): 266-294.
12) Boccia M, Piccardi L, Guariglia P. The Meditative Mind: A Comprehensive Meta-Analysis of MRI Studies. Biomed Res Int. 2015; 419808.
13) Lutz A, Greischar L L, Rawlings N B, Ricard M, Davidson R J. Long-term meditators self-induce high-amplitude gamma synchrony during mental practice. Proc Natl Acad Sci USA. 2004 Nov. 16; 101(46): 16369-16373.
14) The Science of Meditation. Available from: https://samharris.org/podcasts/111-science-meditation/
15) Lagopoulos J, Xu J, Rasmussen I, Vik A, Malhi G S, Eliassen C F, Arntsen I E et al. Increased Theta and Alpha EEG Activity During Nondirective Meditation. The Journal of Alternative and Complementary Medicine, 2009; 15 (11): 1187
16) What are brain waves. Available from: https://brainworksneurotherapy.com/what-are-brainwaves
17) GarcialTArgibay M, Santed M A, Reales J M. Efficacy of binaural auditory beats in cognition, anxiety, and pain perception: a metalTanalysis. Psychological Research. 2018.
18) R. Padmanabhan, A. J. Hildreth, D. Laws. “A prospective, randomised, controlled study examining binaural beat audio and pre-operative anxiety in patients undergoing general anaesthesia for day case surgery”. 2005, https://onlinelibrary.wiley.com/doi/full/10.1111/j.1365-2044.2005.04287.x
19) E. E. Chuter, M. Allan, D. Laws, “A pilot study comparing reduction of anxiety by binaural beat audio and patient-selected music in the pre-operative period”. 2007 https://onlinelibrary.wiley.com/doi/full/10.1111/j.1365-2044.2006.04944_12.x
20) Saxby E, Peniston E G “Alpha-theta brainwave neurofeedback training: an effective treatment for male and female alcoholics with depressive symptoms”. 1995 https://neurofeedbackclinic.ca/journals/depression/dep2.pdf
21) Charles G. Watson, Joseph Harder and Frank T. Passini, “Alpha biofeedback therapy in alcoholics: An 18-month follow-up” 1978, https://onlinelibrary.wiley.com/doi/abs/10.1002/1097-4679%28197807%2934%3A3%3C765%3A %3AAID-JCLP2270340339%3E3.0.CO%3B2-5
22) Billy Cox, “Veterans with PTSD praise audio-therapy results”. Aug. 20, 2015 https://www.orlandosentinel.com/new/os-ap-ptsd-audio-therapy-20150829-story.html
23) Sewak R, Spielholz N I. Relapse prevention: Using sound to reduce the probability of recidivism and suffering following detoxification. Med Hypotheses. 2018; 118:84-91.
24) Science of Sleep. Available from:

https://www.nationalgeographic.com/magazine/2018/08/science-of-sleep/

We claim:

1. A platform for delivering digital behavior therapies to patients, comprising:
a server computer (110);
a database (120) operatively coupled to the server computer;
a mobile patient device (150); and
a therapist device (160) configured to be communicative coupled to the server computer and the mobile patient device;
wherein the mobile patient device comprises a processor, memory, an audio system, and a mobile app that configures the mobile patient device to deliver, via the audio system, selected behavior therapies to a patient, said selected behavior therapies including a selected binaural beat designed to induce a selected brain wave in the patient in combination with a selected set of subliminal affirmations.

2. The platform of claim 1, wherein the selected brain wave and selected binaural beat include one or more of the following: Delta Waves (0.5-3 Hz); Theta Waves (3-8 Hz); Alpha Waves (8-12 Hz); Beta Waves (12-38 Hz); and Gamma Waves (38-100 Hz).

3. The platform of claim 2, wherein the mobile app is configured to selectively provide digital therapies for stress, anxiety, addiction, and depression.

4. A method for delivering digital behavior therapies to a patient, comprising:
providing a platform including a server computer, a database, and a mobile patient device; and
delivering to a patient, via the mobile patient device, selected behavior therapies including a selected binaural beat designed to induce a selected brain wave in the patient in combination with a selected set of subliminal affirmations.

5. The method of claim 4, wherein the selected brain wave and selected binaural beat include one or more of the following: Delta Waves (0.5-3 Hz); Theta Waves (3-8 Hz); Alpha Waves (8-12 Hz); Beta Waves (12-38 Hz); and Gamma Waves (38-100 Hz).

6. The method of claim 4, wherein the platform further comprises a therapist device configured to be communicative coupled to the server computer and the mobile patient device.

7. The method of claim 4, wherein the mobile patient device comprises a processor, memory, an audio system, and a mobile app that configures the mobile patient device to deliver said digital behavior therapies.

8. The method of claim 7, wherein the mobile app is configured to selectively provide digital therapies for stress, anxiety, addiction, and depression.

9. A platform for delivering digital behavior therapies to patients, comprising:
a server computer;
a database operatively coupled to the server computer;
a mobile patient device; and
a therapist device configured to be communicative coupled to the server computer and the mobile patient device;
wherein the mobile patient device comprises a processor, memory, an audio system, and a mobile app that configures the mobile patient device to deliver, via the audio system, selected behavior therapies to a patient, said selected behavior therapies including a selected binaural beat designed to induce a selected brain wave in the patient in combination with a selected set of meditation exercises.

10. The platform of claim 9, wherein the selected brain wave and selected binaural beat include one or more of the following: Delta Waves (0.5-3 Hz); Theta Waves (3-8 Hz); Alpha Waves (8-12 Hz); Beta Waves (12-38 Hz); and Gamma Waves (38-100 Hz).

11. The platform of claim 10, wherein the mobile app is configured to selectively provide digital therapies for stress, anxiety, addiction, and depression.

12. A method for delivering digital behavior therapies to a patient, comprising:
providing a platform including a server computer, a database, and a mobile patient device; and
delivering to a patient, via the mobile patient device, selected behavior therapies including a selected binaural beat designed to induce a selected brain wave in the patient in combination with a selected set of meditation exercises.

13. The method of claim 12, wherein the selected brain wave and selected binaural beat include one or more of the following: Delta Waves (0.5-3 Hz); Theta Waves (3-8 Hz); Alpha Waves (8-12 Hz); Beta Waves (12-38 Hz); and Gamma Waves (38-100 Hz).

14. The method of claim 13, wherein the platform further comprises a therapist device configured to be communicative coupled to the server computer and the mobile patient device.

15. The method of claim 13, wherein the mobile patient device comprises a processor, memory, an audio system, and a mobile app that configures the mobile patient device to deliver said digital behavior therapies.

16. The method of claim 15, wherein the mobile app is configured to selectively provide digital therapies for stress, anxiety, addiction, and depression.
